(19) United States
(12) Patent Application Publication
(10) Pub. No.: US 2003/0158480 A1
(43) Pub. Date: Aug. 21, 2003

(54) Grooved medical devices with enhanced ultrasound visibility

(76) Inventors: Audun Tornes, Oslo (NO); Morten Eriksen, Oslo (NO)

Correspondence Address: AMERSHAM BIOSCIENCES, PATENT DEPARTMENT, 800 CENTENNIAL AVENUE, PISCATAWAY, NJ, 08855, US

(21) Appl. No.: 10/275,213

(22) PCT Filed: Apr. 12, 2001
(86) PCT No.: PCT/GB01/01647

(30) Foreign Application Priority Data
May 15, 2000 (GB) 0011568.3

(51) Int. Cl. A61B 8/00

(52) U.S. Cl. 600/437

(57) ABSTRACT

Medical devices, wherein at least one part of the outer surface of the container is grooved, preferably with a curved groove are disclosed. The grooved outer surface is preferably substantially free from angularities. Such grooves enhance the echogenicity of the device using medical ultrasound at a greater range of angles to the ultrasound probe, thus enhancing the ultrasound visibility of the device.

This invention relates to medical devices for human use. More particularly, it relates to grooved medical devices, with improved ultrasound imaging visibility, i.e. echogenicity.

In clinical practice, it is often vital to the therapeutic outcome for the medical personnel to know the relative position of the medical device in relation to the mammalian tissue. Thus, when a needle, catheter or cannula or other medical device is being used to deliver a drug or therapy, it is important to ensure that the treatment is delivered to the correct tissue. Alternatively, when the medical device (e.g. a biopsy or amniocentesis needle) is being used to take a biological sample from the mammalian body, precise knowledge of the location of the sampling site (i.e. the needle tip) is required.

It is possible to determine a location for each medical device, which will give the desired delivery of therapy. This can be done using a knowledge of the dimensions of the device, an accurate knowledge of the dimensions of the tissue or tissues in relation to which the device is to be placed, plus a knowledge of the position of said tissue relative to a reference point. The dimensions of tissues and organs within the body for use in such determinations may be obtained prior to placement of the medical device using conventional diagnostic imaging techniques including X-ray imaging, magnetic resonance imaging (NM and ultrasound imaging. However, difficulties may arise during the medical device placement procedure which may adversely affect the accuracy of the placement of the device if only pre-placement images are used to guide the device placement. For example, tissue volume may change as a result of swelling or draining of fluid to and from the tissue. Tissue position and orientation can change in the patient's body relative to a selected internal or external reference point as a result of for example manipulation during surgical procedures, movement of the patient or changes in the volume of adjacent tissue. Thus, it is difficult to achieve accurate placement of medical devices to achieve a desired treatment delivery using only knowledge of tissue anatomy and position that was obtained prior to the placement procedure. Therefore, it is advantageous if real-time visualisation or both the tissue and the medical device can be provided. A particularly preferred imaging method due to its safety, ease of use and low cost, is ultrasound imaging.

During the placement of the medical devices into position, the surgeon can monitor the position of tissues such as the prostate gland using, for example, transrectal ultrasound pulse-echo imaging techniques which offer the advantage of low risk and convenience to both patient and surgeon. It is also known to monitor the position of the needle used in implantation procedures using ultrasound. During an implantation or insertion procedure, the location of the tip of the needle is typically monitored.

Ultrasound reflections may be either specular (mirror-like) or scattered (diffuse). Biological tissue typically reflects ultrasound in a scattered manner, whilst metallic devices tend to be effective reflectors of ultrasound. Relatively large smooth surfaces such as those of needles used in medical procedures reflect sound waves in a specular manner. The ultrasound visibility of smaller medical devices such as stents or thermotherapy seeds, is highly dependent upon the angular orientation of the device with respect to the ultrasound transducer used for imaging. The ultrasound reflection from a surface is dependent on the surface shape and can be deduced from diffraction considerations. Thus, a smooth flat surface will generally act as a mirror, reflecting ultrasound waves in the wrong direction unless the angle between the sound and the surface is 90°. A smooth cylindrical structure such as a needle, catheter or cannula will reflect waves in a fan shaped conical pattern pointing away from the transducer, but will only give strong ultrasound reflections when imaged at an angle very close to 90°.

Thus, medical devices with a smooth metallic surface are effective ultrasound reflectors, but the reflected ultrasound intensity is strongly dependent on the orientation of the device with respect to the ultrasound beam. Theory and practical experiments show that even at an angle of 8 degrees between the long axis of a cylindrical device and the ultrasound transducer (i.e. a deviation of 80 from orthogonal), the signal intensity drops by a factor of 100 (20 dB), and the device becomes difficult to detect. At an orientation of 10-12 degrees the device is difficult to detect against a tissue background. Consequently, even very small deviations from orthogonal relative to the incident ultrasound beam cause substantial reductions in the intensity of the echo signal.

There is therefore a continuing need for medical devices with improved ultrasound imaging visibility, and in particular for devices where the dependence of visibility on the angular orientation of the axis of the device with respect to the ultrasound transducer is reduced. Since the total returned echo intensity is limited by the physical size of the device, improvements require broadening the angular range of echo return. The present invention provides medical devices with improved ultrasound visibility, by reducing the angular dependence of the reflected ultrasound.

Efforts have been made to enhance the ultrasound visibility of medical devices (e.g. surgical needles, solid stylets and cannulae) by suitable treatment of their surfaces such as roughening, scoring or etching. Thus, U.S. Pat. No. 4,401,124 discloses a surgical instrument (a hollow needle device) that has a diffraction grating inscribed on the surface to enhance the reflection coefficient of the surface. Sound waves that strike the grooves are diffracted or scattered as secondary wave fronts in many directions, and a percentage of these secondary waves are detected by the ultrasound transducer. The diffraction grating is provided for use at the leading edge of a surgical instrument for insertion within a body, or for use along a surface of an object the position of which is to be monitored while in the body.

U.S. Pat. No. 4,869,259 discloses a medical needle device that has a portion of its surface particle-blasted to produce a uniformly roughened surface that scatters incident ultrasound, such that a portion of the scattered waves is detected by an ultrasound transducer.

U.S. Pat. No. 5,081,997 discloses surgical instruments with sound reflective particles imbedded in a portion of the surface. The particles scatter incident sound, and a portion is detected by an ultrasound transducer.

U.S. Pat. No. 4,977,897 discloses a tubular cannula device comprising a needle and an inner stylet in which one or more holes are cross-drilled perpendicular to the axis of the needle to improve ultrasound visibility. The solid inner stylet may be roughened or scored to enhance the sonographic visibility of the needle/stylet combination.

WO 98/27888 describes a echogenically enhanced medical device in which a print pattern mask of non-conductive epoxy-containing ink is transfer-coated to the surface of the device, flash dried, and then thermally crosslinked. Portions of the needle not protected by the mask are removed by etching in an electropolishing step to leave a pattern of substantially square depressions in the bare metal, and the ink masked is removed with a solvent and mechanical scrubbing. The depressions provide the device with enhanced echogenicity under ultrasound.

U.S. Pat. No. 4,805,628 discloses a device which is inserted or implanted for long-term residence in the body, which device is made more visible to ultrasound by providing a space in the device which has a substantially gas impermeable wall, such space being filled with a gas or mixture of gases. The invention is directed to IUD's (intrauterine devices), prosthetic devices, pacemakers, and the like.

McGahan, J. P., in “Laboratory assessment of ultrasonic needle and catheter visualisation.” Journal of Ultrasound In Medicine, 5(7), 373-7, (July 1986) evaluated seven different catheter materials for their sonographic visualisation in vitro. While five of the seven catheter materials had good to excellent sonographic detection, nylon and polyethylene catheters were poorly visualised. Additionally, various methods of improved needle visualisation were tested. Sonographic needle visualisation was aided by a variety of methods including either roughening or scoring the outer needle or inner stylet and placement of a guide wire through the needle.

WO 00/28554, which is commonly assigned to the present assignee, discloses roughened brachytherapy sources, including seeds, which exhibit enhanced echogenicity. This disclosure shows that the ultrasound visibility of radioactive sources suitable for use in brachytherapy can be improved, even though such sources are relatively much smaller than needles, catheters etc.

Some medical devices, such as thermoseeds or stents, are intended to remain permanently at the site of implantation. However, individual devices may, on rare occasions, migrate within a patient's body away from the initial site of implantation or insertion. This is highly undesirable from a clinical perspective, but any such migration should be readily detectable, e.g. by ultrasound imaging.

Parameters such as the amplitude and shape of surface irregularities and the distance between repeating surface pattern details determine the angular dependency of echo reflections. As part of the present invention, a large number of prototype samples have been evaluated and a narrow range of design options has been identified. A range of surface shapes have been tested: circular and helical sinusoidal and square grooves, triangular grooves, dimples and sandblasted surfaces. Profiles with sharp corners were found to widen the angular range more than smooth shapes. Dimpled surfaces were not found to work as well as grooved surfaces.

According to one aspect of the present invention there is therefore provided a medical device suitable for use in the mammalian body, especially the human body, wherein at least a portion of the outer surface of the device comprises a series of grooves which have:

(i) a depth of 5 to 100 micrometers,

(ii) a width of 200 to 500 micrometers,

(iii) a spacing of 300 to 700 micrometers.

The term “spacing” refers to the distance between the highest points of successive grooves. The grooved surface is optimised to enhance the ultrasound visibility (i.e. the echogenicity) of the device. The design modifications disclosed here involve modifying the outer surface of medical devices. When the device is hollow, optional changes to the inner surface are also described. Increasing the angular range of ultrasound echo reflection from the device is accompanied by a reduction in the overall echo intensity. Hence, the selected design will always be a compromise between signal intensity and strength with respect to the angular orientation. The present invention provides an optimised design for echogenic medical devices for the range of angles of reflection which are of most clinical importance.

The medical devices of the present invention comprise biocompatible materials. Suitable such materials include metals or metal alloys such as titanium, gold, platinum and stainless steel; plastics such as polyesters and vinyl polymers, and polymers of polyurethane, polyethylene and poly(vinyl acetate), composites such as composites of graphite, and glass such as matrices comprising silicon oxide. The plastic or polymer may be coated with a layer of a biocompatible metal. The device may also be plated on the outside with a biocompatible metal, for example titanium, gold or platinum. The device preferably comprises a biocompatible metal on its' outer surface. Titanium and stainless steel are preferred biocompatible metals for the devices.

The medical devices will be of a range of overall size and dimensions depending on the intended use. Thus e.g. the overall dimensions of a catheter for intracoronary use are determined by the size of the coronary artery. For insertion into the coronary artery, the diameter should not exceed about 1 mm, and is preferably about 0.8 mm, most preferably about 0.6 mm.

The term “medical device” means a device which is used for temporary insertion, or for temporary or permanent implantation into the mammalian body, especially of a human patient. Radioactive sources suitable for use in brachytherapy are excluded from the scope of the present invention. Examples of medical devices within the scope of the present invention are: needles (e.g. for implantation, administration, biopsy or amniocentesis); catheters; cannulae; stents; stylets; thermotherapy seeds or “thermoseeds”; RF ablation probes or needles or cryotherapy probes or needles.

As used herein, the term “grooved” means a surface or part surface which is not essentially planar, but which comprises a series of linked raised areas or ridges, and indented areas (or grooves), giving an undulating effect. The grooves may be arranged in a regular pattern or may be random, or there may be present a mixture of random and regular regions. Preferably, the grooves are arranged in a regular pattern, and are preferably of curved cross-section. The resulting grooved outer surface is preferably “substantially free from angularities”. This term implies that the surface undulations are curved in cross-section, i.e. the undulations form a series of smooth curves, with the minimum of angular or sharp edges. The preferred surface is thus approximately sinusoidal or flattened sinusoidal in profile. Typically, the groove width is 10% to 90%, and preferably 40% to 60% of the groove spacing, in the most preferred aspect wherein the grooves are flattened sinusoidal in profile, the groove width is 50% of the groove spacing. Surface shapes which maximise the variance of the container's outer radius are the most acoustically effective shapes, the flattened sinusoidal surface profile is therefore particularly preferred. The raised areas or ridges may themselves be curved outwards (i.e. be convex), or may be planar. Preferably the raised areas or ridges are planar, and are of uniform disposition so that when e.g. the medical device is substantially cylindrical in shape, the raised areas form part of the outer surface of the cylinder. When the grooves are identical, the spacing is in effect the pattern repetition distance.

The term “depth” is the amplitude of the groove, i.e. the vertical distance from the bottom of the groove to the top of the groove. For a given groove depth, which may be limited by design and manufacturing constraints, the flattened sinusoidal surface profile provides better distribution of the reflected ultrasound echo than a pattern consisting of narrow depressions.

The term “spacing” refers to the distance between the highest points of successive grooves. When the grooves are identical, the spacing is in effect the pattern repetition distance.

The term “series of grooves” means one or more grooves. The grooves should be distributed over a sufficient portion of the outer surface of the device, and shaped so that the scattering of ultrasound by the device is adequate for imaging in the range of angles between the ultrasound transducer probe and the device for the given clinical application. The grooves may occur over substantially the entire surface of the device, at one or both ends, in the centre or over any other portion of the outer surface. For a needle, the grooves are preferably located at or near the needle tip, so that the tip is easiest to visualise using ultrasound. This is particularly important when the needle is being used to implant one or more radioactive sources such as seeds for brachytherapy, where the exact location is critical to the radiation dosimetry calculation.

The grooves of the present invention should not exceed 100 μm in amplitude, since when the amplitude is too large destructive interference may occur, and the reflected intensity at orthogonal incidence is dramatically reduced. Preferred grooves have an amplitude or depth of up to approximately one quarter of a wavelength of the ultrasound involved in water—at an ultrasound frequency of 7.5 MHz, this is about 50 μm (50 micrometers or 0.05 mm). The minimum amplitude of the grooves should be at least 5 μm, preferably about one tenth of a wavelength, i.e. 20 to 30 μm. The suitable range for the amplitude of the grooves is therefore 5 to 100 μm typically 15 to 75 μμm, with 20 to 60 μm being preferred and the range 30 to 50 μm being most preferred. In a particularly preferred aspect, the grooves have a nominal depth or amplitude of 45 μm

The grooves may be arranged randomly on the surface of the medical device, or in more regular patterns, for example in geometric shapes and patterns such as concentric circles, or as lines running substantially parallel or perpendicular to an axis of the device e.g. in a circumferential arrangement to give bands or corsets, or in a helical arrangement. Helical or parallel groove patterns are preferred, especially in a band or corseted arrangement. Suitable patterns can be readily determined to suit the exact size and shape of the medical device concerned.

By reducing the distance between repeating surface pattern details, the ultrasound reflection at large angles will increase, but the reflection at small angles will be reduced. Hence, for the typical imaging frequencies used the spacing should be 300 to 700 μm (0.3 to 0.7 mm), preferably 400 to 600 μm (0.4 to 0.6 mm), and most preferably 450 to 550 μm.(0.45 to 0.55 mm). In a particularly preferred aspect, the grooves have a nominal spacing of 500 μm.

The tem groove “width” is the distance measured between the two points on the groove which are at a depth equal to the mean outer radius of the device. Where the grooves have a symmetrical profile such as sinusoidal, preferably flattened sinusoidal, the groove width will be equal to half of the groove spacing.

The groove width in the devices according to the invention is 200 to 500 micrometers. In a preferred aspect, the grooves have a width of 200 to 300 μm, suitably 225 to 275 μm. In a particularly preferred aspect, where the grooves are flattened sinusoidal in profile and the nominal spacing is 500 μm, the grooves have a nominal width of 250 μm.

One advantage of using the devices of the invention is that the ultrasound signal and image may be read, measured and analysed by suitable computer software sufficiently quickly to permit real-time visualisation. This is advantageous from a clinical viewpoint for both patient and medical personnel. However, the devices of the invention may be used in processes involving any type of mapping that uses information obtained due to the ultrasound visibility of the sources. Thus, for implanted devices, imaging may be performed at some time post-implantation to confirm the location of the device.

In addition, a physician may use the same imaging technique, i.e. ultrasound, already in place during surgery to confirm both organ (e.g. prostate) position and size, and medical device placement. This could enable a physician to determine if the relative position of the device within the organ or target of interest needs to be adjusted, based on the actual position of the device.

In a further aspect of the invention, there is provided a method for increasing the ultrasound visibility of a medical device. The method comprises providing the outer surface or part of the outer surface of the container with grooves of specific dimensions and arrangement, effective to enhance reflection of ultrasound to thus facilitate detection of the device in vivo.

In a still further aspect of the invention, there is provided a method for the preparation of a medical device where at least one part of the surface of which is grooved. The grooved surface of the present invention may be produced by a variety of different methods. For example, the outer surface of the device may be grooved by forcing the device through a ridged or serrated die or a threading device. A similar effect may be produced by milling. Parallel grooves or ‘corsets’ may be produced by a crimping process using a die tool set. The die set is produced by electrode sparking or etching duplicate sets or grooves into pieces or machined steel, or by high precision milling. The two dies are then polished to a mirror finish so that they meet precisely once pressed together. The device is inserted into the grooved area of the die set, and the two dies brought together, thus introducing grooves into the surface of the device. The depth of the grooves obtained is controlled by the pressure applied.

One or more helical grooves may also be produced by gently pressing a sharp metal edge to the surface of a device while the container is rolled over a solid surface at a slight angle. Spiral or helical grooves can be introduced using two tools which fit together allowing a gap the same diameter as the device. Across the face of one tool is a diagonal raised profile which is the same shape and size as the desired groove. The device is rolled between the tools and the diagonal profile inscribes a helical or spiral groove across the device as it rotates. The surface of the tools used must be roughened or coated to provide sufficient friction to the device, enabling it to roll as the tools move.

The grooved surface may also be achieved by etching, for example using a laser or water-jet cutter, or by electrolytic etching. Blasting, for example sand blasting, may also be used. Such blasting may be done dry, or wet as in water-jet blasting.

When the medical device comprises a hollow cylinder or tube designed to transfer material within its' walls, (e.g. a needle, cannula or catheter), it is preferred that the grooves are introduced in such a way (e.g. compression, crimping or related techniques), that the transfer of material through the device is not impaired. Thus, the inner surface of the wall preferably remains substantially smooth, and the internal diameter of the cylinder preferably remains constant. This permits efficient transfer of material either to or from the mammalian body. This can be achieved by techniques which result in only the outer surface being grooved, whereas the inner surface remains essentially planar. Suitable such techniques are compression, where the amount of wall material remains the same, or removal of wall material to form the groove.

It is also envisaged that, certain types of grooves on the inner surface of hollow devices designed to transfer materials, may facilitate the transfer of material. Thus, e.g. longitudinal or spiral grooves on the inside of a needle, cannula or similar may function to improve fluid transfer in a manner similar to the rifling of gun barrels. When the device is in the form of an ultrasound reflective inner material (e.g. metal), coated with a material which has comparable ultrasound transmission characteristics to water or mammalian tissue (e.g. organic polymers), then it is envisaged that the outer surface of such a device could be planar, and only the inner surface carry the grooves of the present invention.

When the medical device comprises a biocompatible metal, e.g. titanium, it is also preferred that the metal is annealed prior to any mechanical working, compression etc. of the metal. Annealing is known to those skilled in the art, and involves heating the metal to a high temperature below its melting point, followed by slow cooling back to ambient temperature either in vacuo or in an inert atmosphere, typically of argon. These precautions prevent any surface oxidation or other reaction between the hot metal and the surrounding atmosphere (e.g. nitride formation). For titanium, general annealing is carried out at 400-750° C., more preferably at 700±50° C. or at 25-55° C. below the beta transus temperature of 913±15° C. for recrystallisation annealing. Such annealed metals are more amenable to working, i.e. mechanical manipulation, reshaping etc., since they exhibit reduced risk of introducing weaknesses such as microfractures into the metal when the metal is subjected to stress.

When the medical device is hollow (e.g. a needle or catheter), the thickness of the wall may preferably be within the specifications set for conventional medical devices. This is not inconsistent with the teaching that the depth of the surface grooves can be up to 60 μm, since the inner surface can be grooved as described above.

The medical device may optionally be provided with more than one type of groove. These may take the form of different depths, spacings, shapes or patterns, e.g. different parallel grooves (or corsets) or different advancing spiral or helical threads (which may be in the same or opposite sense of handedness), either alone or in combination.

The external grooves of the present invention may facilitate transmission or insertion of the medical device by presenting a reduced outer surface area for frictional resistance to the outer contact surface of the needle or cannula, or by a rifling effect, when the grooves are generally in the same direction as the direction of movement of the device.

The invention will be further illustrated, by way of example, with reference to the following Figures:

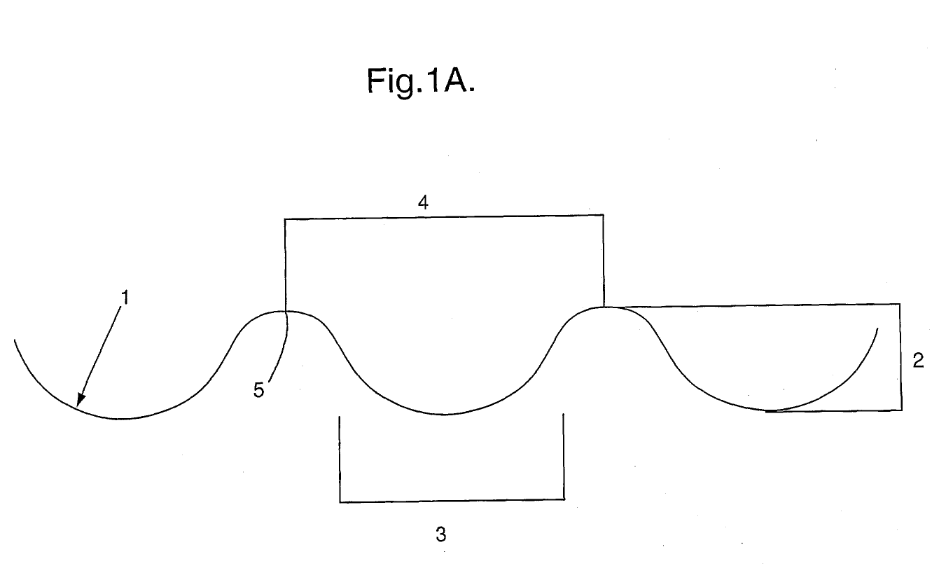

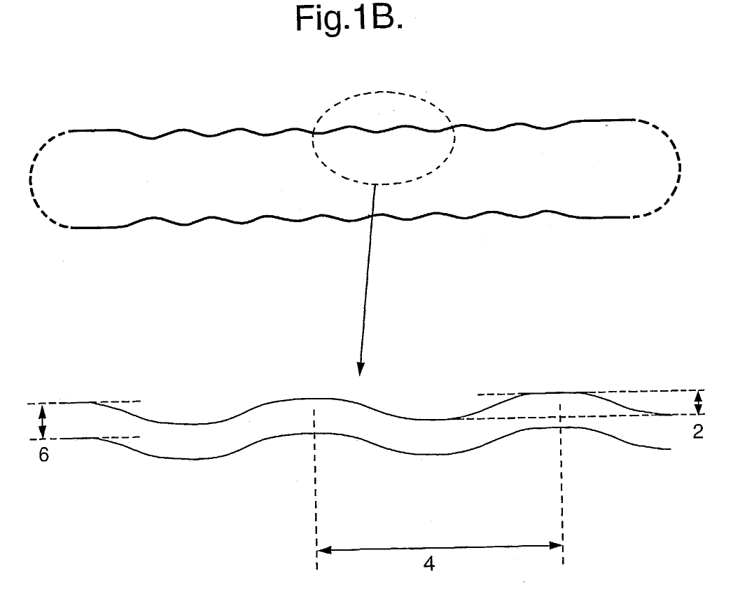

FIG. 1A illustrates the medical device outer surface according to the invention;

FIG. 1B shows an expanded view of a grooved medical device wall having both internal and external grooves according to the invention;

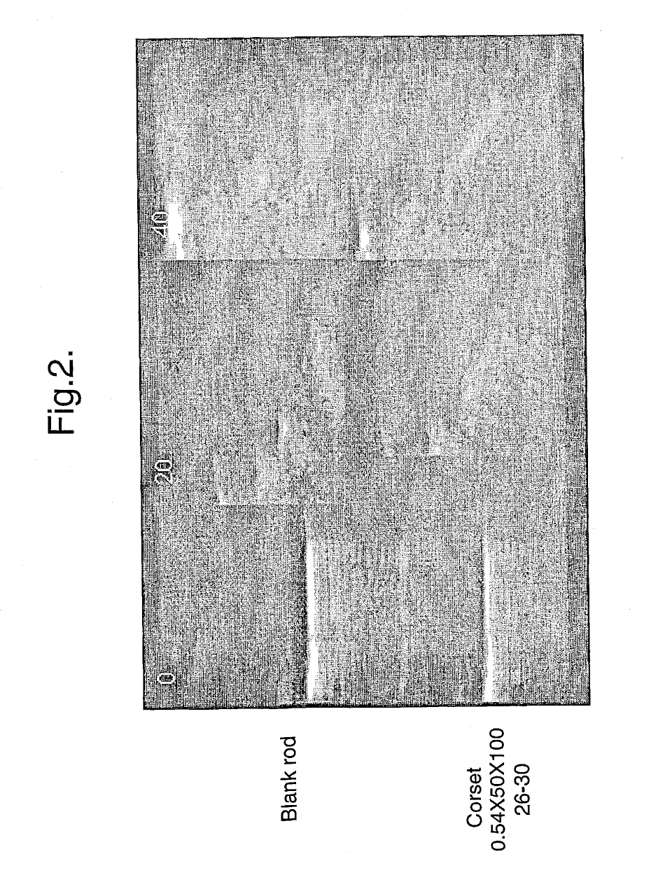

FIG. 2 compares ultrasound images from grooved and ungrooved steel rods at 0, 20 and 40 degrees from orthogonal;

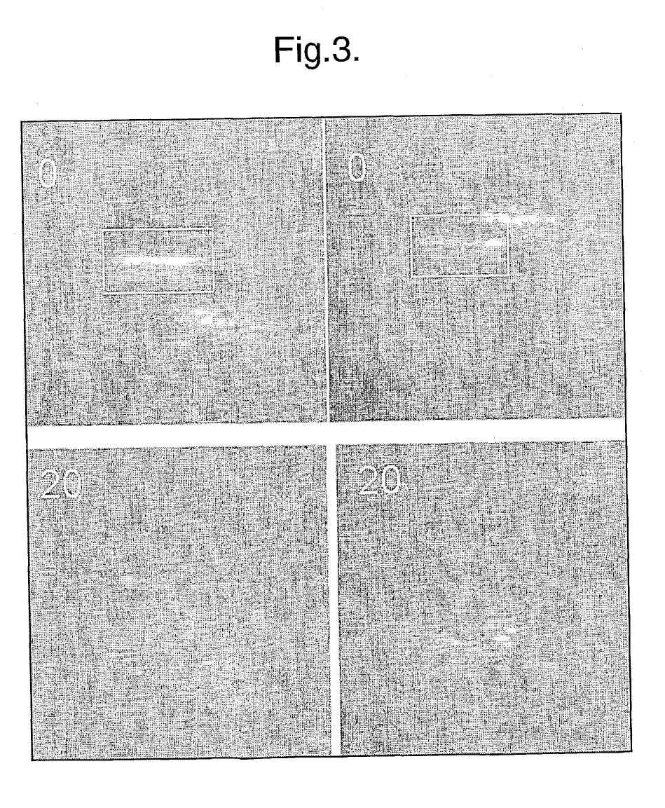

FIG. 3 compares ultrasound images from grooved and ungrooved steel rods in excised dog prostate tissue;

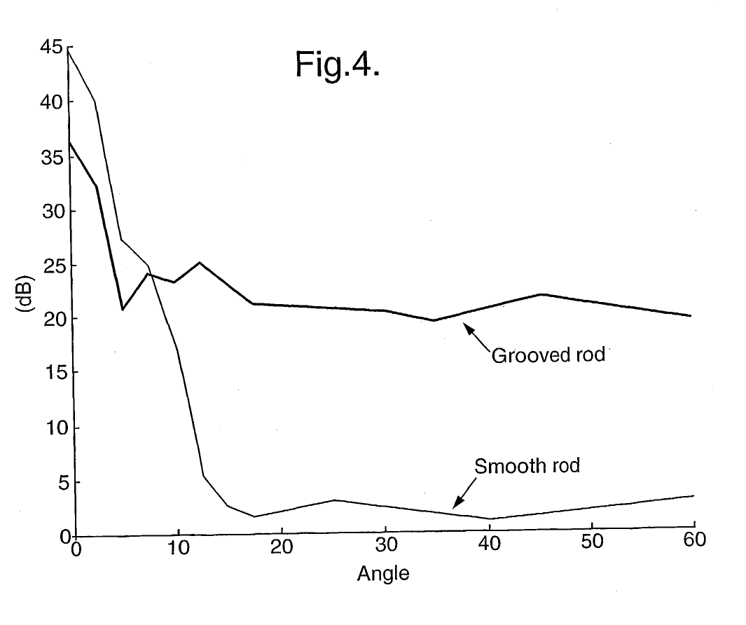

FIG. 4 compares the reflected ultrasound signal intensity at various angles of reflection from grooved and ungrooved steel rods;

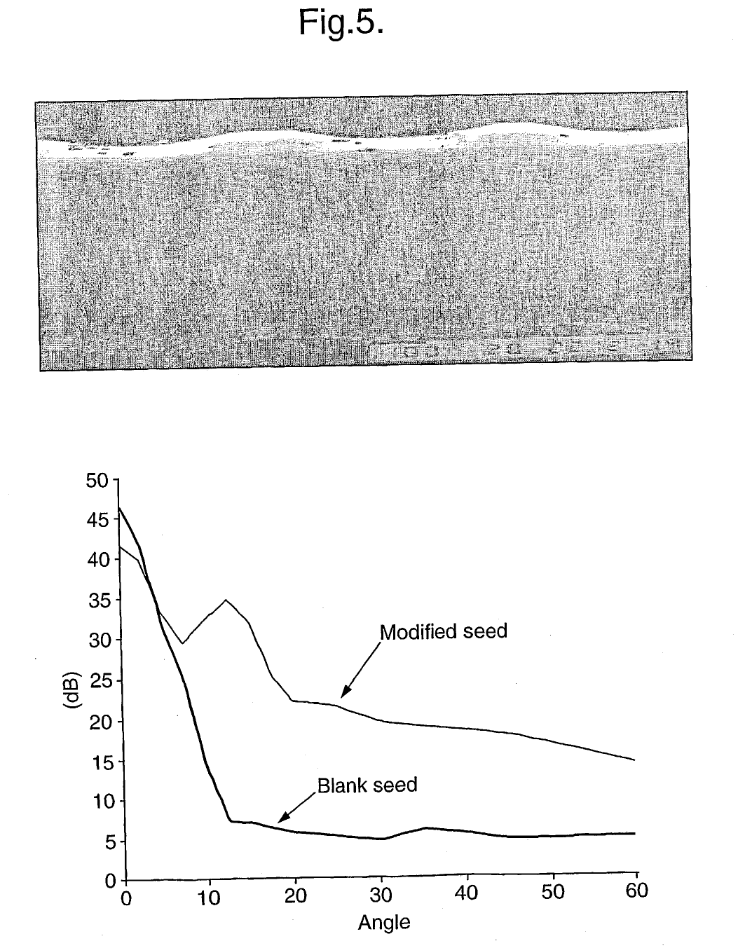

FIGS. 5 compares the ultrasound signal intensity from a grooved hollow titanium canister (image shown) with a corresponding ungrooved titanium canister, at various angles;

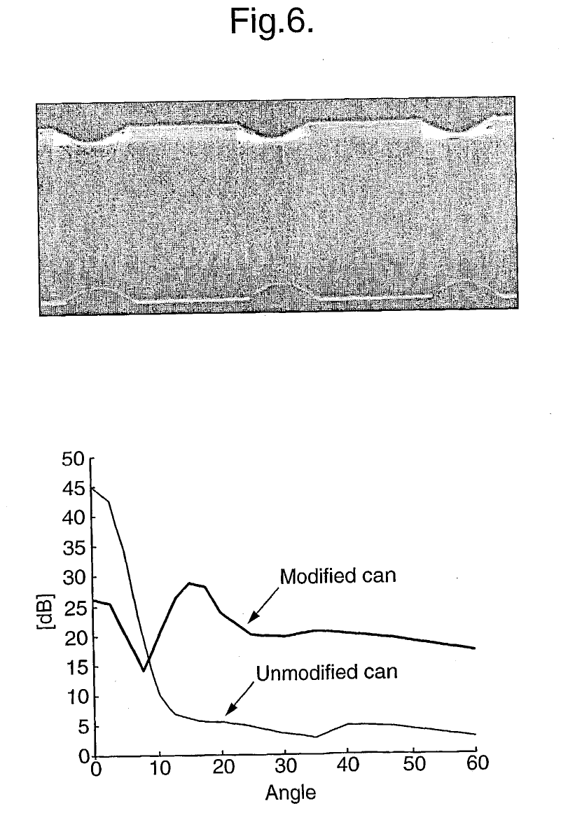

FIG. 6 compares the ultrasound signal intensity from an angular grooved steel rod with a smooth rod;

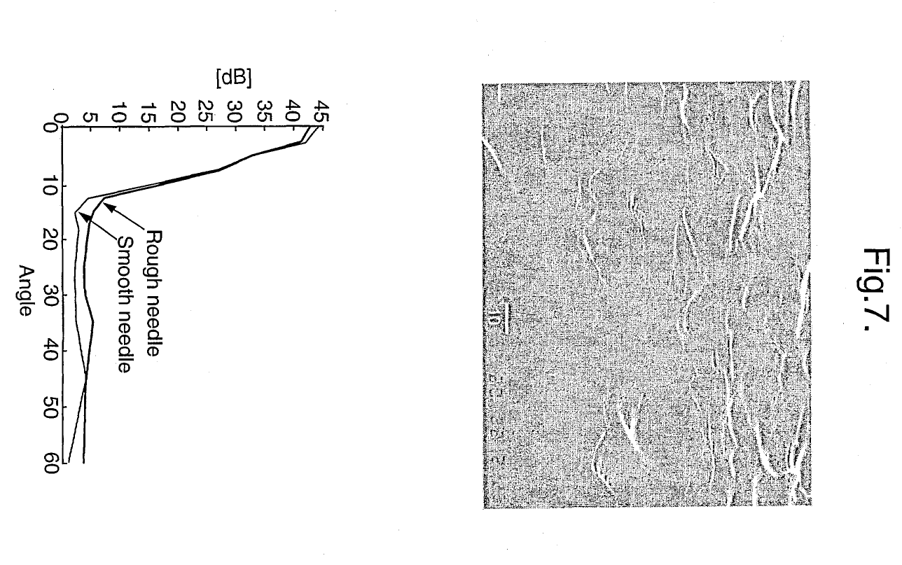

FIG. 7 compares the ultrasound signal intensity for needles with smooth and randomly roughened surfaces.

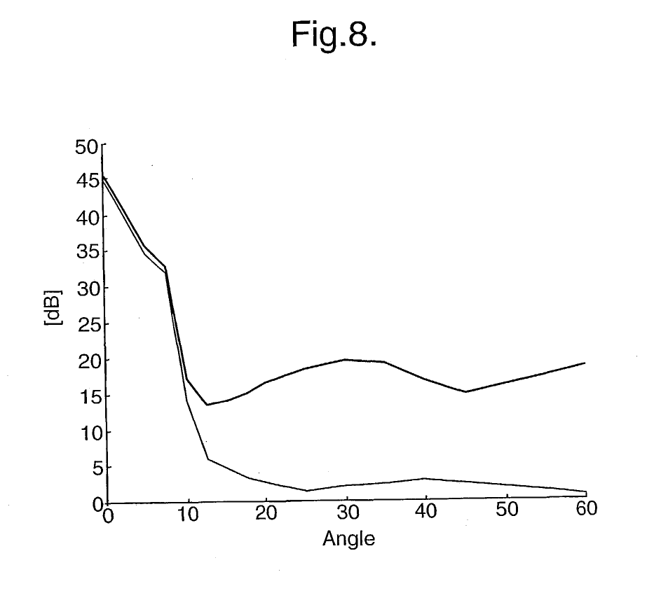

FIG. 8 compares the ultrasound signal intensity from a commercially available echogenic needle tip with that of the smooth part of the needle.

The invention will be further illustrated with reference to the following non-limiting

### EXAMPLES

### Example 1

A wide band imaging ultrasound transducer ATL L10-5 was mounted in the wall of a water tank. The transducer was connected to an ATL HDI 5000 ultrasound scanner and imaging was performed at 6.5 MHz, a typical imaging frequency used in clinical transrectal ultrasound.

The test object (a hollow titanium canister) was mounted on a holder located 50 mm from the transducer surface, which could be rotated to defined angles in relation to the direction of the ultrasound beam. The canister was glued on to the tip of a needle protruding from the specimen holder with cyanoacrylate glue so that the canister's centre of gravity coincided with the rotational axis of the holder. The angular rotation could be set with half a degree accuracy, which is of great importance given the high angular dependency of the ultrasound backscatter. The holder could also be adjusted by translation to position the canister in the focal point of the transducer and fixed throughout the experiments.

A series of measurements mapping the ultrasound backscatter of each of the test objects throughout the full range of incidence angles (−65 to +65 degrees) were performed. Digital images were stored for quantitative analysis of echo signal intensity with a custom-made image analysis system. An angular reflection index was defined as the range of angles where the echo signal is above a threshold defined to be 20 dB below the maximum signal intensity of a smooth surface test object at orthogonal incidence.

### Example 2

A smooth steel rod and a rod with a square surface pattern was imaged in vitro as described in Example 1. Images are acquired at 0, 20 and 40 degrees rotation, and are shown in FIG. 2. The upper series of images is a smooth 0.8 mm diameter, 6.5 mm length steel rod while the lower series is a similar steel rod with a cut surface with 0.1 mm wide helical square grooves, having a spacing of 0.54 mm and with a depth of 0.05 mm.

### Example 3

An excised dog prostate was imaged in a water tank with an ATL HDI 5000 scanner using an imaging frequency of 6.5 MHz. Two steel rods as described in Example 2 were implanted using an 18G needle. The prostate with the rods implanted was then rotated and imaged at different angles—see FIG. 3.

### Example 4

Two 0.8 mm diameter, 6.5 mm length solid steel rods, one with a smooth surface and one with helical square grooves as in Examples 2 and 3 (pitch 0.54 mm, width 0.1 mm and depth 0.05 mm) were imaged at different rotational angles as described in Example 1. The signal intensity from the centre of the rods were measured and plotted against angle—see FIG. 4.

### Example 5

Two 0.8 mm diameter, 6.5 mm length titanium canisters, one with a smooth surface, and one with a sinusoidal helical surface pattern, were imaged at different rotational angles as described in Example 1. The signal intensity from the centre of the canisters was measured and plotted against angle. The sinusoidal surface pattern had a groove amplitude of 0.04 mm and a spacing/pitch of 0.5 mm. The results are shown in FIG. 5.

### Example 6

Two 0.8 mm diameter, 6.5 mm length steel rods, one with a smooth surface and one with a circular square surface pattern were imaged at different rotational angles as described in Example 1. The signal intensity from the centre of the canisters were measured and plotted against angle. The circular square groove pattern had an amplitude of 0.070 mm, width of 0.2 mm and a spacing/pitch of 0.5 mm. The results are shown in FIG. 6.

### Example 7 (Comparative Example)

Two needles, one with a smooth surface and one with a sandblasted surface pattern were imaged at different rotational angles as described in Example 1. The signal intensity from the centre of the needles was measured and plotted against angle—see FIG. 7, which shows the roughened needle (upper line) and smooth needle (lower line). The sandblasted surface had randomly spaced irregularities, see image of FIG. 7. The irregularities are in the size range of up to about 20 microns.

### Example 8 (Comparative Example)

A commercially available echogenic needle (Cook brachytherapy needle CBD 018020), having a diamond pattern surface etching echotip was imaged at different rotational angles as described in Example 1. A smooth part of the needle was imaged for reference. The signal intensity from the centre of the needles was measured and plotted against angle—the results are shown in FIG 8.

### Example 9

### (Annealing Procedure)

A titanium pipe 500 mm long and of 20 mm diameter, fitted with an argon supply (99.99% purity, flow 5 dm³/min) at one end was used. The pipe was flushed with argon for 30 min prior to loading. Non-radioactive, sealed titanium canisters of dimensions equivalent to seeds, were loaded into a porcelain ship, which was then introduced into the open end of the pipe. The pipe was inserted into a pre-heated electric furnace with thermocouple temperature control maintained at 700° C. The pipe was kept in the furnace for 30 min (15 min to heat to 700° C., and 15 min at 700° C.), then the oven was switched off and the pipe and dummy seeds allowed to cool to ambient whilst maintaining the argon atmosphere.

Comparison of FIG. 5 with FIGS. 7 and 8 (prior art), shows that the grooves of the present invention provide significantly enhanced echogenicity over a wider range of angles than prior art roughened needles.

FIG. 1A is a schematic illustration of part of a device surface (not to scale), with grooves [1]. The amplitude or depth [2] of the grooves is 20 to 60 micrometers. The width [3] of the grooves is 200 to 500 micrometers, and the groove spacing [4] is in the range 300 to 700 micrometers. The ridges [5] extend to the outer surface of the source, and may be convex (as shown), or substantially planar.

FIG. 1B is an expanded view of a preferred grooved hollow device wall design, showing a grooved inner and outer wall surface (where the device may be open-ended or closed as shown with dotted lines). The spacing [4], depth [2] and uniform wall thickness [6] are shown.

FIG. 2 shows that at 20 and 40 degrees of rotation of the rod relative to the incident ultrasound energy, only the ends of the smooth rods are visible, whilst the fall length of the grooved rod is visible for 0, 20 and 40 degrees rotation.

FIG. 3 (left panels) shows an image of a blank (i.e. ungrooved or smooth) steel rod, and the right panel shows a steel rod with a grooved surface as in Example 2 (0.1 mm wide helical square grooves spaced at 0.54 mm and with a depth of 0.05 mm). The Example shows clearly that the cut rod is more visible than the blank rod at 20 degrees of rotation.

FIG. 4 shows that the reflected signal intensity from the grooved rod (upper line) is weaker than that of the smooth rod (lower line) at small angles, but much stronger for angles above about 10 degrees.

FIG. 5 shows that the reflected signal intensity from the modified sinusoidal shaped grooved surface of a hollow titanium canister (upper line), is somewhat weaker than that of the smooth canister (lower line) at small angles, but significantly stronger for angles above about 10 degrees.

FIG. 6 shows that the reflected signal intensity from a circular square grooved metal rod (upper line), is much weaker than that of a smooth rod at small angles, but stronger for large angles.

FIG. 7 shows an image of a sandblasted needle surface, having random roughening, and compares the reflected ultrasound signal intensity for this needle versus a smooth needle. The acoustic effect of this modified surface is seen to be much smaller than for the optimised grooves of the present invention.

FIG. 8 compares the reflected ultrasound signal intensity for a commercially available echogenic needle tip with that of the smooth part of the needle. The acoustic effect of this modified surface is seen to be much smaller than for the optimised grooves of the present invention.

1. A medical device suitable for human use, wherein at least a portion of a surface of the device comprises a series of grooves which have:
(i) a depth of 5 to 100 micrometers,
(ii) a width of 200 to 500 micrometers,
(iii) a spacing of 300 to 700 micrometers.

2. The medical device of claim 1, wherein the grooves are on the outer surface.

3. The medical device of claim 1 or 2, wherein the grooves are of curved cross-section.

4. The medical device of any of claims 1 to 5, wherein the depth is 30 to 50 micrometers.

5. The medical device of any of claims 1 to 4, where the spacing is 450 to 550 micrometers.

6. The medical device of any of claims 1 to 5 wherein the groove width is 225 to 275 micrometers.

7. The medical device of any of claims 1 to 6 wherein the surface is provided with circular circumferential grooves.

8. The medical device of claims 1 to 7, which comprises an annealed biocompatible metal.

9. The medical device of claims 1 to 8, which is a needle.

10. The medical device of claims 1 to 8, which is a catheter.

11. The medical device of claims 1 to 8, which is a stent.

12. The medical device of claims 1 to 8, which is a thermoseed.

13. The medical device of claims 1 to 8, which is a cannula.
